SEQUENCE LISTING (8 sequences)

SEQ ID NO: 1 - protein, 560 aa, Human immunodeficiency virus type 1
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVL

SEQ ID NO: 2 - DNA, 9229 bp, Human immunodeficiency virus type 1
ggtctctctggttagaccagatttgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggaggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcagccaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcagctacactagaagaaatgatgacagcatgtcagggagtgggaggacccggccataaggcaagagttttggctgaagcaatgagccaagtaacaaattcagctaccataatgatgcaaagaggcaattttaggaaccaaagaaagattgttaagtgtttcaattgtggcaaagaagggcacatagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccatttcttcagagcagaccagagccaacagccccaccagaagagagcttcaggtctggggtagagacaacaactccctctcagaagcaggagccgatagacaaggaactgtatcctttaacttccctcagatcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagtcctattgaaactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtgggaaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaacgaggggtgcccacactaatgatgtaaaacaattaacagaggcagtgcaaaaaataaccacagaaagcatagtaatatggggaaagactcctaaatttaaactacccatacaaaaggaaacatgggaaacatggtggacagagtattggcaagccacctggattcctgagtgggagtttgtcaatacccctcctttagtgaaattatggtaccagttagagaaagaacccatagtaggagcagaaacgttctatgtagatggggcagctagcagggagactaaattaggaaaagcaggatatgttactaatagaggaagacaaaaagttgtcaccctaactgacacaacaaatcagaagactgagttacaagcaattcatctagctttgcaggattcgggattagaagtaaatatagtaacagactcacaatatgcattaggaatcattcaagcacaaccagataaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatactttcttttaaaattagcaggaagatggccagtaaaaacaatacatacagacaatggcagcaatttcaccagtactacggttaaggccgcctgttggtgggcgggaatcaagcaggaatttggaattccctacaatccccaaagtcaaggagtagtagaatctatgaataaagaattaaagaaaattataggccaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatctgggtcagggagtctccatagaatggaggaaaaagagatatagcacacaagtagaccctgaactagcagaccaactaattcatctgtattactttgactgtttttcagactctgctataagaaaggccttattaggacatatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccatttcagaattgggtgtcgacatagcagaataggcgttactcaacagaggagagcaagaaatggagccagtagatcctagactagagccctggaagcatccaggaagtcagcctaaaactgcttgtaccacttgctattgtaaaaagtgttgctttcattgccaagtttgtttcacaacaaaagccttaggcatctcctatggcaggaagaagcggagacagcgacgaagacctcctcaaggcagtcagactcatcaagtttctctatcaaagcagtaagtagtacatgtaatgcaacctatacaaatagcaatagcagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttggggaatgctactaataccaatagtagtaataccaatagtagtagcggggaaatgatgatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttgaatggcagtctagcagaagaagaggtagtaattagatctgccaatttcacagacaatgctaaaaccataatagtacagctgaaccaatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagcggacaaattagatgttcatcaaatattacagggctgctattaacaagagatggtggtaataacaacaatgggtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcacggtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacattccttaattgaagaatcgcaaaaccagcaagaaaagaatgaacaagaattattggaattagataaatgggcaagtttgtggaattggtttaacataacaaattggctgtggtatataaaaatattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtggttggatggcctactgtaagggaaagaatgagacgagctgagccagcagcagatggggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatttgagcctgggagctctctggctaactagggaacccactgcttaagcctcaataaagcttgccttgagtgcttca

SEQ ID NO: 3 - DNA, 36 bp, Artificial Sequence
  OLA primer 1 for detection of Q509L
agactcacaatatgcattaggaatcattcaagcaca

SEQ ID NO: 4 - DNA, 36 bp, Artificial Sequence
  OLA primer 2 for detection of Q509L
agactcacaatatgcattaggaatcattcaagcact

SEQ ID NO: 5 - DNA, 40 bp, Artificial Sequence
  OLA primer 3 for detection of Q509L
attatttgattgactaactctgattcacttttatctggta

SEQ ID NO: 6 - DNA, 40 bp, Artificial Sequence
  OLA primer 4 for detection of Q509L
attatttgattgactaactctgattcacttttatctggtt

SEQ ID NO: 7 - DNA, 31 bp, Artificial Sequence
  Virus PCR Forward primer
aagctataggtacagtattagtaggacctac

SEQ ID NO: 8 - DNA, 28 bp, Artificial Sequence
  RT PCR Reverse Primer
tgctctccaattactgtgatatttctca

(19) United States
(12) Patent Application Publication
(10) Pub. No.: US 2007/0196902 A1
(43) Pub. Date: Aug. 23, 2007

(54) Method of detecting AZT resistance in HIV

(75) Inventors: John W. Mellors, Pittsburgh, PA (US); Dianna Koontz, Pittsburgh, PA (US); Jessica H. Brehm, Pittsburgh, PA (US)

Correspondence Address: JESSE A. HIRSHMAN, ESQ., 1722 MURRAY AVENUE, THIRD FLOOR, PITTSBURGH, PA, 15217, US

(73) Assignee: University of Pittsburgh, Pittsburgh, PA (US)

(21) Appl. No.: 11/699,941

(22) Filed: Jan. 30, 2007

Related U.S. Application Data
(60) Provisional application No. 60/763,627, filed on Jan. 31, 2006.

(51) Int. Cl. C12Q 1/68; C12P 19/34; C12N 5/06; C12N 5/16

(52) U.S. Cl. 435/91.1; 435/345; 435/6

(57) ABSTRACT

Described herein is a method of determining the presence of a nucleoside reverse transcriptase inhibitor-resistant Human Immunodeficiency Virus-1 (HIV-1) virus particle in a biological sample, comprising identifying the presence in the sample of a point mutation at codon Q509 of an HIV-1 reverse transcriptase, for example and without limitation Q509L. That point mutation increases resistance to AZT about 3- to 10-fold by itself and about 50-fold in combination with the connection domain mutation A371V.

### CROSS REFERENCE TO RELATED APPLICATIONS

This application claims the benefit under 35 U.S.C. §119(e) to U.S. Provisional Patent Application No. 60/763,627, filed Jan. 31, 2006, which is incorporated herein by reference in its entirety.

### STATEMENT REGARDING FEDERAL FUNDING

This invention was made with government support under NIH NCI SAIC Subcontract No. 20XS190A. The government has certain rights in this invention.

Human immunodeficiency virus type 1 (HIV-1) reverse transcriptase (RT) is responsible for the conversion of the viral single-stranded RNA genome into double stranded DNA. The enzyme is multifunctional and exhibits both RNA and DNA-dependent DNA polymerase activity and RNase H activity. FIGS. 1A and 1B provide the GenBank consensus protein and nucleotide sequence of a reverse transcriptase of a Human immunodeficiency virus 1 (FIG. 1A shows amino acids 588-1147 of GenBank Accession No. NP_{—}057849 (“p66 subunit” protein, SEQ ID NO: 1) and FIG. 1B shows GenBank Accession No. K02013 (nucleic acid, SEQ ID NO: 2, in which bases 2132 to 3811 encode the polypeptide of FIG. 1A). HIV-1 RT is a heterodimer composed of a 560 amino acid 66 kDa subunit (p66) and a p66-derived 440 amino acid 51 kDa subunit (p51). The p66 subunit contains both the DNA polymerase and RNase H active sites and is composed of three domains: the N-terminal polymerase domain (residues 1-318 of SEQ ID NO: 1); the connection domain (residues 319-426 of SEQ ID NO: 1); and the C-terminal RNase H domain (residues 427-560 of SEQ ID NO: 1). By contrast, the p51 subunit is composed of only the polymerase and connection domains and may largely play a structural role in RT heterodimer stability.

HIV-1 RT is an important therapeutic target, and two distinct groups of RT inhibitors have been identified. These include the nucleoside or nucleotide reverse transcriptase inhibitors (NRTI), which include zidovudine (3′-azido-3′-dideoxythymidine, AZT), lamivudine (3TC), emtrictabine (FTC), zalcitabine (ddC), didanosine (ddI), stavudine (d4T), abacavir (ABC) and tenofovir (TNV), and the nonnucleoside inhibitors (NNRTI), which include nevirapine, delavirdine and efavirenz. NRTI inhibit HIV-1 replication by competing with the natural dNTP substrate for binding and incorporation into the nascent DNA chain. Once incorporated, NRTI act as DNA chain-terminators. By contrast, NNRTI bind to RT in a location distinct from the polymerase active site and act as allosteric inhibitors of HIV-1 reverse transcription. Although combination therapies that contain two or more RT inhibitors have profoundly reduced morbidity and mortality from HIV-1 infection, their long-term efficacy is limited by the selection of drug-resistant variants of HIV-1.

Mutations that confer resistance to NRTIs have been identified by in vitro passage experiments and from sequences amplified from patients experiencing virologic failure on NRTI therapy. In general, NRTI-associated resistance mutations can be broadly categorized into two groups depending on their mechanism of resistance (Marcelin, A. G., C. Delaugerre, M. Wirden, P. Viegas, A. Simon, C. Katlama, and V. Calvez. 2004. Thymidine analogue reverse transcriptase inhibitors resistance mutations profiles and association to other nucleoside reverse transcriptase inhibitors resistance mutations observed in the context of virological failure. J. Med. Virol. 72:162-5 and Sluis-Cremer, N., D. Arion, and M. A. Parniak. 2000. Molecular mechanisms of HIV-1 resistance to nucleoside reverse transcriptase inhibitors (NRTIs). Cell Mol. Life. Sci. 57:1408-22). The polymerase domain mutations M41L, D67N, K70R, L210W, T215F/Y and K219Q/E are typically referred to as thymidine analog mutations (TAMs). These mutations increase the ability of HIV-1 RT to excise a chain-terminating NRTI-monophosphate (NRTI-MP) from a prematurely terminated DNA chain (Arion, D., N. Kaushik, S. McCormick, G. Borkow, and M. A. Parniak. 1998. Phenotypic mechanism of HIV-1 resistance to 3′-azido-3′-deoxythymidine (AZT): increased polymerization processivity and enhanced sensitivity to pyrophosphate of the mutant viral reverse transcriptase. Biochemistry 37:15908-17; Boyer, P. L., S. G. Sarafianos, E. Arnold, and S. H. Hughes. 2001. Selective excision of AZTMP by drug-resistant human immunodeficiency virus reverse transcriptase. J. Virol. 75:4832-42; Meyer, P. R., S. E. Matsuura, A. M. Mian, A. G. So, and W. A. Scott. 1999. A mechanism of AZT resistance: an increase in nucleotide-dependent primer unblocking by mutant HIV-1 reverse transcriptase. Mol. Cell. 4:35-43; and Sarafianos, S. G., A. D. Clark, Jr., K. Das, S. Tuske, J. J. Birktoft, P. Ilankumaran, A. R. Ramesha, J. M. Sayer, D. M. Jerina, P. L. Boyer, S. H. Hughes, and E. Arnold. 2002. Structures of HIV-1 reverse transcriptase with pre- and post-translocation AZTMP-terminated DNA. EMBO J 21:6614-24). This resistance mechanism has been termed NRTI excision. By comparison, the polymerase domain mutations K65R, K70E, L74V, Q151M (in complex with A62V, V751, F77L, and F116Y) and M184V increase the selectivity of RT for incorporation of natural dNTP substrate versus the NRTI-triphosphate (NRTI-TP) (Huang, H., R. Chopra, G. L. Verdine, and S. C. Harrison. 1998. Structure of a covalently trapped catalytic complex of HIV-1 reverse transcriptase: implications for drug resistance. Science 282:1669-75; Krebs, R., U. Immendorfer, S. H. Thrall, B. M. Wohrl, and R. S. Goody. 1997. Single-step kinetics of HIV-1 reverse transcriptase mutants responsible for virus resistance to nucleoside inhibitors zidovudine and 3TC. Biochemistry 36:10292-300; Sarafianos, S. G., K. Das, A. D. Clark, Jr., J. Ding, P. L. Boyer, S. H. Hughes, and E. Arnold. 1999. Lamivudine (3TC) resistance in HIV-1 reverse transcriptase involves steric hindrance with beta-branched amino acids. Proc. Natl. Acad. Sci. USA 96:10027-32; and Sluis-Cremer, N., P. Argoti Torres, J. Grzybowski, U. Parikh, and J. Mellors. 2006. Presented at the 13th Conference on Retroviruses and Opportunistic Infections, Denver, Colo.). This resistance mechanism has been termed NRTI discrimination.

### SUMMARY

Described herein are point mutations identified as conferring drug resistance to HIV. In one embodiment, a previously unrecognized point mutation in the RNAse H domain of the reverse transcriptase of human immunodeficiency virus type 1 (HIV-1) is identified that confers resistance to 3′-azidothymidine (AZT, also known as zidovudine or RETROVIR®). The mutation is located at codon 509 (Q509, e.g., Q509L) of reverse transcriptase in the RNAse H domain. The mutation increases resistance to AZT about 3- to 10-fold by itself and about 50-fold in combination with the connection domain mutation A371V. Because the mutation relates to AZT resistance, it is likely of clinical significance.

Provided therefore, according to one non-limiting embodiment of the methods described herein is a method of determining the presence of a nucleoside reverse transcriptase inhibitor-resistant Human Immunodeficiency Virus-1 (HIV-1) virus particle in a biological sample. The method comprises identifying the presence in the sample of a point mutation at codon 509 of an HIV-1 reverse transcriptase. In one non-limiting embodiment, the point mutation is Q509L. In another embodiment, the method comprises determining the presence in the biological sample of a point mutation at one or more of codons 67, 70, 215 and 371 of an HIV-1 reverse transcriptase, such as, without limitation, one or more of the point mutations M41L, D67N, K70R, T215I, T215F, T215Y and A371V in an HIV-1 reverse transcriptase. In one further embodiment, the method comprises determining the presence in the biological sample of the point mutations D67N, K70R and Q509L in an HIV-1 reverse transcriptase. Specific examples of combinations of point mutations correlating with increased AZT resistance are provided in Tables 4 and 5.

According to a further non-limiting embodiment, nucleoside reverse transcriptase inhibitor is one of 3′-azido-3′-deoxythymidine, stavudine, didanosine, zalcitabine, lamivudine, abacavir and emtricitabine. In another, the nucleoside reverse transcriptase inhibitor is 3′-azido-3′-deoxythymidine.

Any method of determining the presence of the point mutation(s) may be employed in the methods described herein, including, without limitation one or more of: sequencing of a cDNA or an amplification product thereof, allele-specific PCR, Oligonucleotide Ligation assay, clonal analysis and Single Genome Sequencing. In one non-limiting example, the method comprises preparing cDNA from HIV-1 RNA in the sample and sequencing at least a portion of the cDNA or an amplification product thereof to determine the presence of the point mutation in the reverse transcriptase. In one version of that method, a portion of the cDNA comprising a sequence encoding codon 509 of the reverse transcriptase protein is amplified and sequenced. In another version, a portion of the cDNA comprising a sequence encoding codons 41 through 509 of the reverse transcriptase protein is amplified and sequenced in order to determine the presence of mutations in any of codons 41-509, including, without limitation, one or more of codons 41, 67, 70, 215 and 371. The cDNA may be amplified in any useful manner, such as by PCR or isothermic methods, such as strand displacement amplification, loop-mediated isothermal amplification (LAMP), rolling circle DNA amplification, and nucleic acid sequence-based amplification).

Also provided, in order to implement AS-PCR or OLA methods for detection of Q509L, is an isolated nucleic acid comprising at its 3′ terminus one of the sequences: 5′-ttcaagcaca-3′ (SEQ ID NO: 3, nucleotides 27-36), 5′-ttcaagcact-3′ (SEQ ID NO: 4, nucleotides 27-36), 5′-ttatctggta-3′ (SEQ ID NO: 5, nucleotides 31-40) or 5′-ttatctggtt-3′ (SEQ ID NO: 6, nucleotides 31-40). The nucleic acid may be fluorescently-labeled, for instance with a fluorescein, Cy 3, Cy5 dye. In one embodiment, the nucleic acid comprises at its 3′ terminus 10 or more contiguous nucleotides of the 3′ end of one of the sequences:

(SEQ ID NO: 3)

5′-agactcacaatatgcattaggaatcattcaagcaca-3′,

(SEQ ID NO: 4)

5′-agactcacaatatgcattaggaatcattcaagcact-3′,

(SEQ ID NO: 5)

5′-attatttgattgactaactctgattcacttttatctggta-3′,
or

(SEQ ID NO: 6)

5′-attatttgattgactaactctgattcacttttatctggtt-3′.

### BRIEF DESCRIPTION OF THE DRAWINGS

FIGS. 1A and 1B provide the consensus protein and nucleotide sequence of a reverse transcriptase of a Human immunodeficiency virus 1 (amino acids 588-1147 of GenBank Accession No. NP_{—}057849_(FIG. 1A, “p66 subunit”, protein, SEQ ID NO: 1) and GenBank Accession No. K02013 (FIG. 1B, nucleic acid, SEQ ID NO: 2)).

FIGS. 2A and 2B describe single-cycle and multiple-cycle replication assays of recombinant HIV_{LAI} containing the A371V and Q509L mutations. FIG. 2A: single-cycle replication was measured in P4/R5 cells infected with 10 ng p24 in a 96 well plate. After 48 hours, cells were lysed and viral replication was measured using RLU. FIG. 2B: multiple-cycle replication was measured in MT-2 cells infected with 10 ng p24. p24 was measured from cell-free supernatant harvested daily for 7 days. Recombinant viruses analyzed: wild-type xxLAI (closed circle), D67N/K70R (open square), D67N/K70R/A371V/Q509L (closed square), D67N/K70R/T215I/A371V/Q509L (open circle) and D67N/K70R/T215F/A371V/Q509L (closed triangle). Data represents the mean±SD from 3 independent experiments. *p-values<0.05 were considered significant compared to wild-type control (xxLAI).

FIGS. 3A and 3B provide structural representations of AZT selected mutations in the p66 subunit of RT. FIG. 3A: location of residues A371 and Q509 in relation to TAMs D67, K70 and T215, the polymerase active site and the RNase H active site. FIG. 3B: both A371 and Q509 are located near the T/P DNA binding tract. A371 is 2.8 Å from K374, whose side chain interacts with the phosphate backbone of the RNA template strand (dotted line). Q509 is in proximity to 1505, a residue that makes up the RNase H primer grip. p66 subunit of RT: cartoon; DNA Primer: black ribbon; RNA Template: grey ribbon. Structure drawn using MOE, based on coordinates from Sarafianos et al., 2001, EMBO J 20:1449. Pdb access number: 1HYS.

### DETAILED DESCRIPTION

Described herein are point mutations identified as conferring drug resistance to HIV. This discovery illustrates the value of including the domains (connection and RNAse H) in which these mutations occur in future genotypic and phenotypic analyses for drug resistance. In one embodiment, a previously unrecognized point mutation in the RNAse H domains of the reverse transcriptase of human immunodeficiency virus type 1 (HIV-1) is identified that confers resistance to 3′-azidothymidine (AZT, also known as zidovudine or RETROVIR®). The mutation is located at codon Q509 (Q509L) of reverse transcriptase in the RNAse H domain. The mutation increases resistance to AZT about 3- to 10-fold by a new mechanism. Because the mutation relates to AZT resistance, it is likely of clinical significance.

The mutation arose in response to AZT and is therefore likely of clinical significance. The mutation arose in the reverse transcription domain, which is not included in current genotypic and phenotypic analyses of drug resistance. This suggests that current analyses, which do not look at mutations in the RNaseH region, for example codon 509, are inaccurate and provide an under-approximation of the incidence and degree of drug resistance in HIV infected patients by current diagnostic measurements. As such, these findings argue for increasing diagnostic analyses of HIV drug resistance to cover the RNAse H domain of the reverse transcriptase of HIV.

It is anticipated that point mutations at codon 509 will confer drug resistance to other nucleoside reverse transcriptase inhibitor (NRTI) drugs, which are a major component of anti-HIV therapy. The newly-identified mutations in HIV-1 reverse transcriptase were identified by selection of AZT resistant virus in vitro and sequencing of the entire reverse transcriptase gene. Then, the mutations were introduced by site-directed mutagenesis into a wildtype infectious molecular clone and virus produced from this clone was phenotyped, demonstrating AZT resistance (see below).

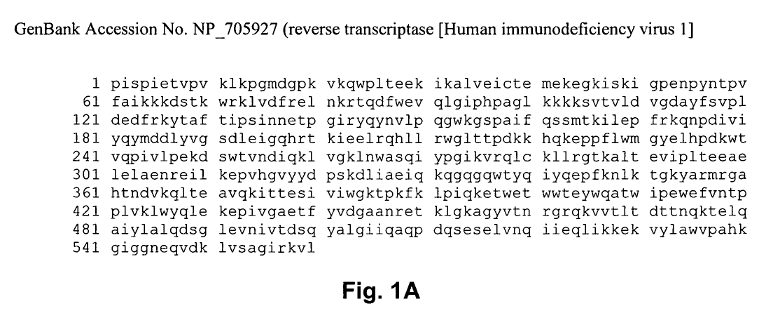

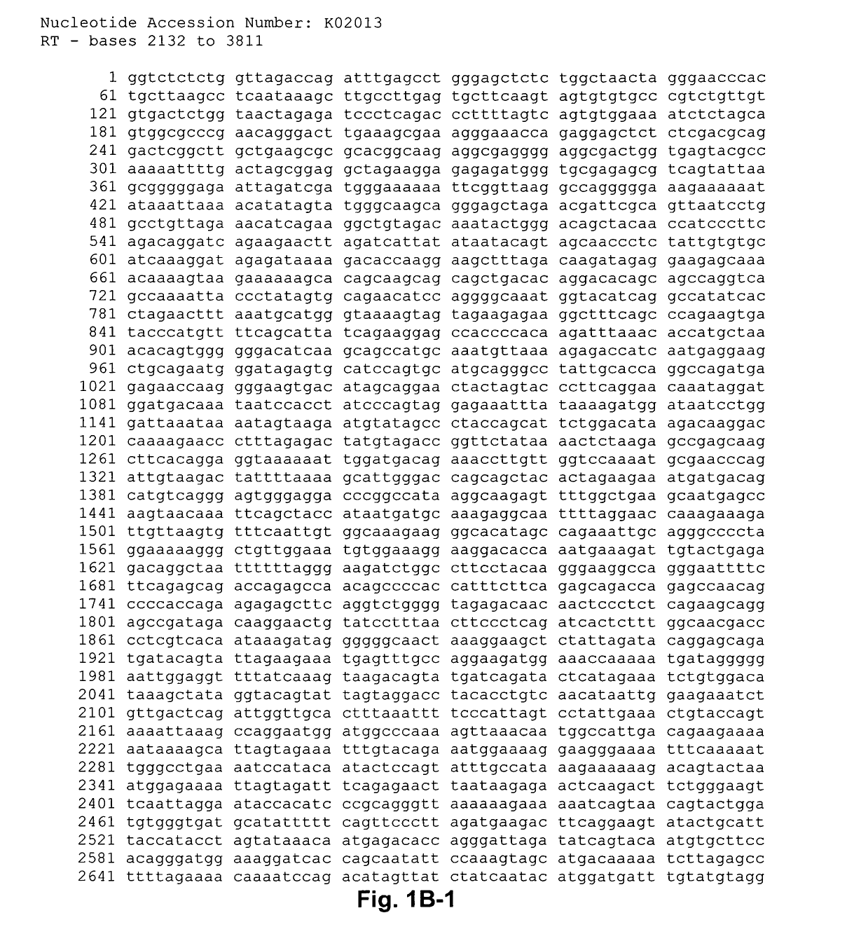

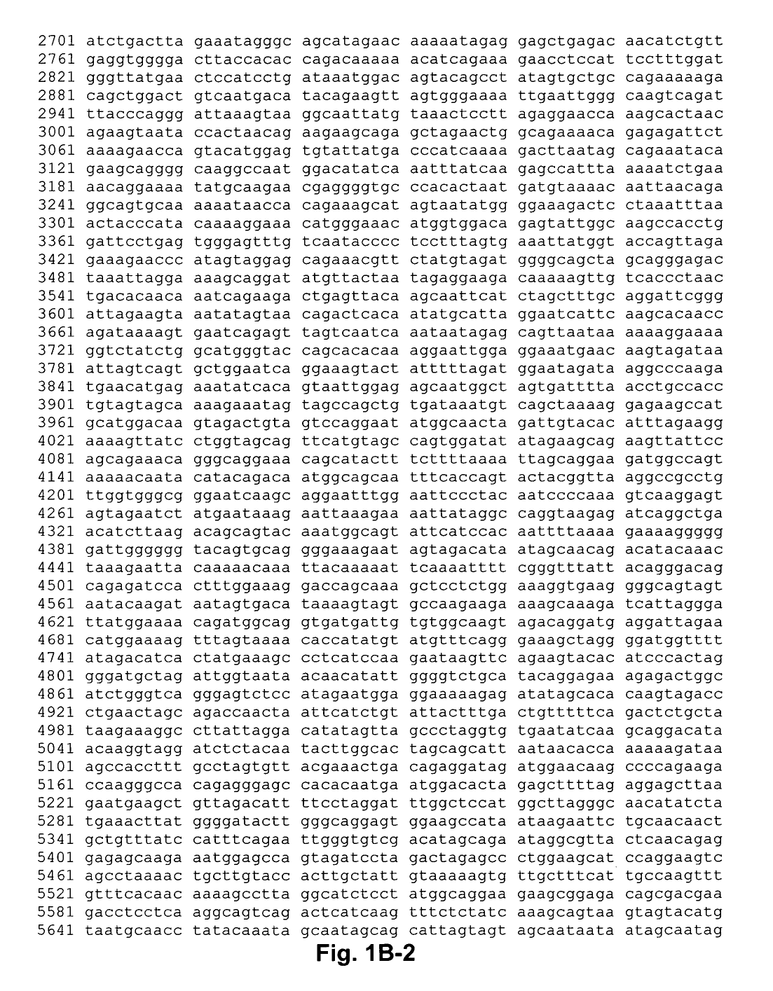

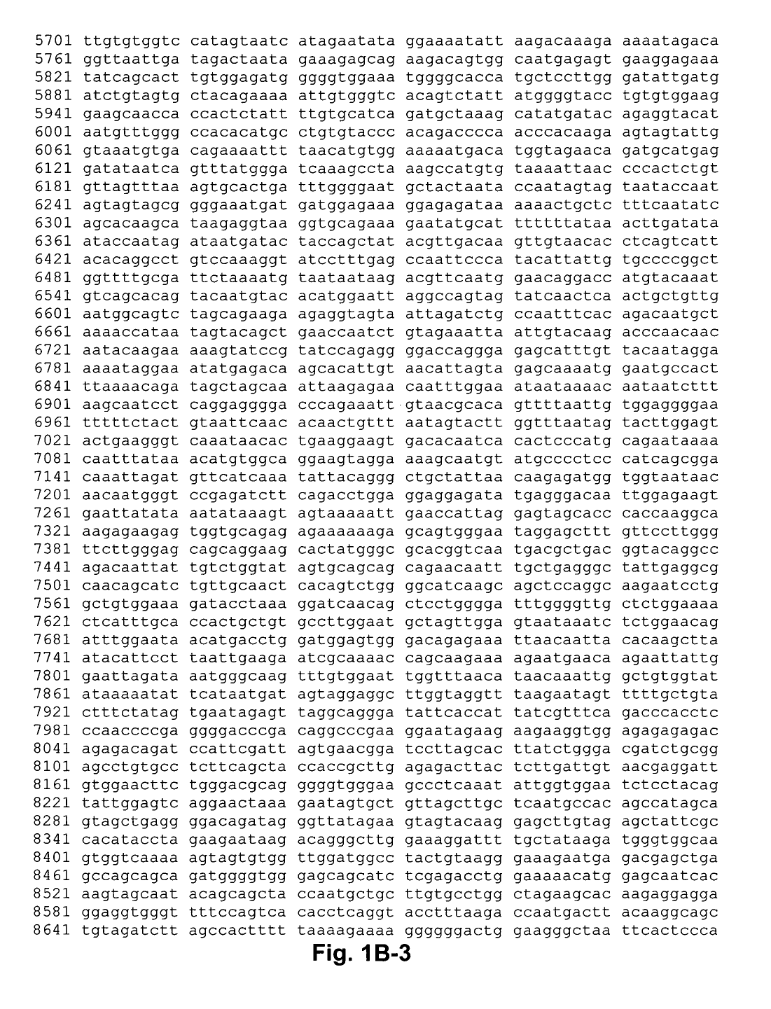

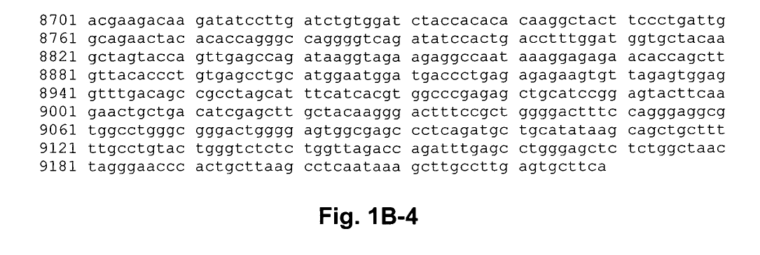

All point mutations described herein are described in comparison to the consensus HIV-1 reverse transcriptase protein sequence provided in FIG. 1A (SEQ ID NO: 1). For nomenclature purposes, point mutations are described as follows: AA₁###AA₂, wherein AA_is the wild-type amino acid at amino acid No. ### in a reference sequence (e.g., SEQ ID NO: 1) and AA2 is the amino acid that replaces the wild-type amino acid. Although the mutations are described herein in reference to a specific sequence (e.g., Q509 and/or Q509L of SEQ ID NO: 1), the point mutation, and not the specific location is what is indicated and intended. For instance, the numbering of codon 509 is in reference to the mature RT protein shown in FIG. 1A. When considering the pre-processed gag-pol protein provided in Genbank Accession No. NP_{—}057849, the RT sequence of FIG. 1A is bases 588-1147 of that sequence. Thus, the point mutation referenced herein as Q509L is Q1096L in reference to the numbering of Genbank Accession No. NP_{—}057849. The point mutation Q1096L in reference to the numbering of Genbank Accession No. NP_{—}057849 is considered to be the mutation Q509L (and thus a mutation at codon 509) for purposes herein.

As indicated above, provided, according to one non-limiting embodiment of the methods described herein is a method of determining the presence of a nucleoside reverse transcriptase inhibitor-resistant Human Immunodeficiency Virus-1 (HIV-1) virus particle in a biological sample. By “biological sample,” it means any sample that comprises or potentially comprises an HIV-1 virus particle, an HIV-1 genome, and/or a portion of an HIV-1 genome, or a nucleic acid copy of an HIV-1 genome or portion thereof, including DNA and/or RNA, such as cDNA and/or amplified DNA or RNA (amplicons). The definition of biological samples is intended to be broadly interpreted as any sample that can contain a nucleic acid and/or protein in which one or more point mutations can be identified in a protein and/or a nucleic acid encoding a protein, including, without limitation: blood, plasma, lymph, urine, sputum, saliva, stool, semen, mucus, cerebrospinal fluid, lavages, biopsies, cell culture medium, purified HIV-1 viral preparations and extractions, including RNA, DNA and/or protein preparations prepared from any of the above.

The method comprises determining the presence in the sample of a point mutation at amino acid 509 of an HIV-1 reverse transcriptase, including portions thereof that contain the point mutation. A point mutation is the substitution of any amino acid for an amino acid of a wild-type protein, such as the substitution of the Gln at amino acid 509 of a wild-type HIV-1 reverse transcriptase protein sequence, as shown by example in FIG. 1A, with Leu residue. Determining the presence of a point mutation in reference to any protein includes any method useful in determining the presence of the point mutation in a biological sample, including protein and nucleic acid sequencing methods as well as affinity methods (e.g., antibody or ligand binding) and hybridization methods (e.g., molecular beacon probing methods).

In one non-limiting embodiment, the point mutation is Q509L. In another embodiment, the method comprises determining the presence in the biological sample of a point mutation at codon 509 along with any other TAM, including without limitation, point mutations in one or more of codons 41, 67, 70, 215 and 371 of an HIV-1 reverse transcriptase, such as, without limitation, one or more of the point mutations M41L, D67N, K70R, T215I, T215F, T215Y and A371V in an HIV-1 reverse transcriptase. Specific combinations of point mutations are described herein, including, without limitation: A371V/Q509L; D67N/K70R/Q509L; D67N/K70R/A371V/Q509L; D67N/K70R/T215I/Q509L; D67N/K70R/T215I/A371V/Q509L; D67N/K70R/T215F/Q509L and D67N/K70R/T215F/A371V/Q509L.

The nucleoside reverse transcriptase inhibitor (NRTI) can be any NRTI, for example and without limitation one of AZT, stavudine, didanosine, zalcitabine, lamivudine, abacavir and emtricitabine. In one example, the nucleoside reverse transcriptase inhibitor is AZT.

Any method of determining the presence of the point mutation(s) may be employed in the methods described herein, including, without limitation: sequencing (standard sequencing), allele-specific PCR, Oligonucleotide Ligation assay (OLA), clonal analysis, Single Genome Sequencing (SGS).

Standard sequencing methods employ, for example and without limitation, direct sequencing PCR product amplified from cDNA prepared from virus samples. In one non-limiting embodiment, HIV RNA is purified from patient plasma, followed by RT-PCR and direct sequencing of the HIV reverse transcriptase coding region, for example and without limitation, by dideoxy termination and variations thereof, for example and without limitation using automated fluorescent dideoxy terminator chemistry. Amplification products of the cDNA also may be sequenced according to standard methods. An amplification product includes amplicons prepared by PCR methods as well as amplification products obtained by use of other amplification methods, such as isothermic methods including without limitation: strand displacement amplification, Loop-Mediated Isothermal Amplification (LAMP), Rolling Circle DNA Amplification, Nucleic Acid Sequence-Based Amplification.

Allele-Specific PCR (AS-PCR) employs, for example and without limitation selective PCR amplification of one of the alleles to detect sequence polymorphisms, for example, Single Nucleotide Polymorphisms (SNP). Selective amplification is usually achieved by designing a primer such that the primer will match/mismatch one of the alleles at the 3′-end of the primer.

Oligonucleotide Ligation assay (OLA, ligase chain reaction) is a method of detecting, for example and without limitation, single base mutations. A primer is synthesized in two fragments and annealed to the template with possible mutation at the boundary of the two primer fragments. The 5′ or upstream fragment typically contains an allele-specific base at its 3′ end, so that template mismatches will not result in ligation. Ligase will ligate the two fragments if they match exactly to the template sequence. Subsequent PCR reactions will amplify only if the primer is ligated. In one non-limiting embodiment, the upstream probe containing the match or mismatch is fluorescently-labeled and genotype-specific. The method may employ two different upstream probes, one for each allele containing two distinguishable fluorescent reporters. The two probes differ in sequence(s) at the last base at their 3′ ends. Allele discrimination occurs by the ability of DNA ligase to join perfectly matched probes; a 3′ mismatch in the capture probe will prevent ligation. Depending on the allele, the PCR products will fluoresce differently.

Clonal analysis includes, without limitation, cloning PCR amplified viral cDNA into a vector (for example and without limitation, a TOPO® clone, Invitrogen Corporation of Carlsbad, Calif.) and sequencing individual clones by standard sequencing methods.

Single Genome Sequencing (SGS) methods include, for example and without limitation, diluting HIV-1 cDNA so that positive PCR reactions arise from one cDNA molecule, then sequencing the PCR product by any useful method.

In non-limiting examples, a portion of the cDNA comprising a sequence encoding (at a minimum) codon 509 of the HIV-1 reverse transcriptase protein is amplified and sequenced using standard sequencing, clonal analysis and/or SGS. In other embodiments, AS-PCR and/or OLA is used to identify point mutations at codon 509 of HIV-1 reverse transcriptase. In yet another embodiment, a portion of the cDNA comprising a sequence encoding codons 41 through 509 of the reverse transcriptase protein is amplified and sequenced, or AS-PCR and/or OLA are employed to determine the presence of mutations in any of codons 41-509, including, without limitation, one or more of codons 41, 67, 70, 215 and 371.

Useful in implementing the methods described herein are isolated nucleic acid comprising at its 3′ terminus one of the sequences 5′-ttcaagcaca-3′ (SEQ ID NO: 3, nucleotides 27-36), 5′-ttcaagcact-3′ (SEQ ID NO: 4, nucleotides 27-36), 5′-ttatctggta-3′ (SEQ ID NO: 5, nucleotides 31-40) or 5′-ttatctggtt-3′ (SEQ ID NO: 6, nucleotides 31-40). Wherein the residue “t” can be a “u” where appropriate. The nucleic acid may be fluorescently labeled. In another embodiment, the isolated nucleic acid comprises 10 or more contiguous nucleotides of the 3′ end of one of the sequences:

(SEQ ID NO: 3)

5′-agactcacaatatgcattaggaatcattcaagcaca-3′,

(SEQ ID NO: 4)

5′-agactcacaatatgcattaggaatcattcaagcact-3′,

(SEQ ID NO: 5)

5′-attatttgattgactaactctgattcacttttatctggta-3′,
or

(SEQ ID NO: 6)

5′-attatttgattgactaactctgattcacttttatctggtt-3′.

These nucleic acids find use in OLA and AS-PCR assays for detection of the mutation Q509L.

### EXAMPLES

All the NRTI mutations included in the most widely used resistance tables, such as that from the IAS-USA expert panel (Johnson, V. A., F. Brun-Vezinet, B. Clotet, B. Conway, D. R. Kuritzkes, D. Pillay, J. M. Schapiro, A. Telenti, and D. D. Richman. 2005. Update of the drug resistance mutations in HIV-1: Fall 2005. Top HIV Med. 13:125-31), are located in the DNA polymerase domain of HIV-1 RT. This is the case, in part, because most commercial genotypic assays do not analyze the complete connection and RNase H domains of RT. In this regard, Nikolenko et al. reported that the mutations D549N and H539N that decrease RT RNase H activity also increase resistance to AZT (Nikolenko, G. N., S. Palmer, F. Maldarelli, J. W. Mellors, J. M. Coffin, and V. K. Pathak. 2005. Mechanism for nucleoside analog-mediated abrogation of HIV-1 replication: balance between RNase H activity and nucleotide excision. Proc. Natl. Acad. Sci. USA 102:2093-8). Specifically, the D549N and H539N mutations increased AZT resistance by 12-fold and 180-fold, respectively, and reduced d4T susceptibility by 2,4-fold and 10-fold, respectively (Nikolenko, G. N., et al. 2005 Proc. Natl. Acad. Sci. USA 102:2093-8). Furthermore, when D549N was present with TAMs D67N, K70R, T215Y and K219Q, AZT and d4T resistance increased 1,230-fold and 12.5-fold, respectively. The mutations had no effect on susceptibility to efavirenz or to ddI and 3TC. The authors proposed that mutations in the RNase H domain that decrease RNase H activity, reduce RNA template degradation, thereby increasing the time for AZT-MP to be excised from the terminated primer and polymerization to resume on an intact template.

It is not known, however, whether mutations in the RNase H domain of RT are selected by AZT. Therefore, we carried out in vitro selections of AZT-resistant HIV-1, sequenced the entire coding region of RT to identify all drug-resistance related mutations, and characterized the effects of these mutations using site-directed recombinant viruses.

Recent work has suggested that mutations in the C-terminal domains of human immunodeficiency virus type 1 (HIV-1) reverse transcriptase (RT) increase 3′-azido-3′-dideoxythymidine (AZT) resistance. Because it is not known if AZT selects mutations outside of the polymerase domain of RT, the following in vitro experiments were carried out in which HIV-1_{LAI} was passaged in MT-2 cells in increasing concentrations of AZT. The first resistance mutations to appear were 2 polymerase domain thymidine analog mutations (TAMs)—D67N and K70R—that together conferred 66-fold AZT resistance. These were followed by the acquisition of 2 novel mutations—A371V in the connection domain and Q509L in the RNase H domain—that in combination with D67N and K70R were associated with ˜90-fold AZT resistance. Thereafter, the T215I mutation appeared but was later replaced by T215F, resulting in a large increase in AZT resistance (˜16,000-fold). The roles of A371V and Q509L in AZT resistance were confirmed by site-directed mutagenesis: A371V and Q509L together increased AZT resistance ˜50-fold in combination with TAMs. Mutagenesis studies also showed that HIV-1 containing D67N/K70R/T215F/A371V/Q509L conferred greater cross-resistance to lamivudine, abacavir and tenofovir than viruses without A371V/Q509L. Taken together, these results provide the first evidence that mutations in the connection and RNase H domains of RT are selected by AZT in combination with TAMs and confer significantly greater AZT resistance and cross-resistance to other nucleoside inhibitors.

Materials and Methods

NRTI. AZT and ddI were obtained from Sigma Chemical Corporation (St. Louis, Mo.). 3TC and d4T were provided by Raymond Schinazi (Emory University). TNV was provided by Gilead Sciences (Foster City, Calif.) and ABC by GlaxoSmithKline (Research Triangle Park, N.C.). NRTIs were prepared as 10 mM or 30 mM stock solutions in dimethyl sulfoxide or sterile water and stored at −20° C. The compounds were diluted immediately before use to desired concentrations in Dulbecco's modified Eagle medium, Phenol Red Free (DMEM-PRF, Gibco-BRL, Grand Island, N.Y.).

Cells and viruses. MT-2 cells (AIDS Research and Reference Reagent Program, National Institute of Allergy and Infectious Diseases, National Institutes of Health) were cultured in RPMI 1640 (Whittaker MA Bioproducts, Walkersville, Md.) supplemented with 10% fetal bovine serum (FBS), 2 mM L-glutamine, 10 mM HEPES buffer, 50 IU/ml of penicillin and 50 mg/ml of streptomycin (referred to as R10). The P4/R5 reporter cell line (provided by Dr. Nathaniel Landau, Salk Institute, La Jolla, Calif.), which expresses the β-galactosidase gene under the control of the HIV long terminal repeat promoter that is transactivated by HIV-1 tat, was maintained in DMEM-PRF supplemented with 10% FBS, 50 IU/ml of penicillin, 50 μg/ml of streptomycin and 0.5 μg/ml of puromycin (Clontech, Palo Alto, Calif.). Stock viruses were prepared in MT-2 cells as described previously (Parikh, U. M., D. L. Koontz, C. K. Chu, R. F. Schinazi, and J. W. Mellors. 2005. In vitro activity of structurally diverse nucleoside analogs against human immunodeficiency virus type 1 with the K65R mutation in reverse transcriptase. Antimicrob. Agents Chemother. 49:1139-44). Briefly, 5 to 10 μg of plasmid DNA was electroporated into 1.3×10⁷ MT-2 cells. Cell-free supernatants were collected 7 days after transfection at peak cytopathic effect (CPE) and stored at −80° C. The infectivity of the virus stocks was determined by a threefold endpoint dilution in P4/R5 cells, and the 50% tissue culture infectivity dose (TCID₅₀) was calculated using the Reed and Muench equation (Reed, L. J., and H. Muench. 1938. A Simple Method of Estimating Fifty Per Cent Endpoints. Am. J. Hyg. 27:493-497). To confirm the genotype of the stock viruses, viral RNA was extracted from cell-free supernatants and treated with 1 Unit/μl of DNase I for 2 hours. Codons 1-560 of RT were amplified using the following primers:

RT forward

(SEQ ID NO: 1, bases 26-58)

5′-AAGCTATAGGTACAGTATTAGTAGGACCTAC-3′;
and
RT reverse

(SEQ ID NO: 3)

5′-TGCTCTCCAATTACTGTGATATTTCTCA-3′.

PCR products were purified (Wizard PCR purification system; Promega, Madison, Wis.) and sequenced using a Big Dye terminator kit (v.3.1) on an ABI 3100 automated DNA sequencer (Applied Biosystems, Foster City, Calif.).

Selection of AZT-resistant viruses. Resistant virus was selected in two independent experiments by the passage of wild-type HIV_{LAI} or HIV_{LAI} containing the M41L/L210W/T215Y mutations (AZT^{R} HIV_{LAI}) in MT-2 cells in increasing concentrations of AZT. To initiate each selection experiment, MT-2 cells (1×10⁶) were pretreated for two hours with 0.5 μM and 25 μM AZT for wild-type HIV_{LAI} and AZT^{R} HIV_{LAI}, respectively, before virus was added. Viral replication was monitored by CPE. At +3/4 CPE (3 or 4 syncytia per field at 100× magnified field), cell-free supernatant was harvested and 0.1 ml of supernatant was added to fresh MT-2 cells to initiate a new passage. The concentration of AZT was doubled every three passages. The selection pressure was increased from an initial AZT concentration of 0.5 μM to a final concentration of 32 μM for wild-type HIV_{LAI}, and from 25 μM to 150 μM for the AZT^{R} HIV_{LAI}. The concentration of drug required to inhibit viral replication by 50% (IC₅₀) was calculated every five passages to identify changes in AZT susceptibility, and fold-resistance was determined by dividing the IC₅₀ of the mutant virus by the IC₅₀ of wild-type HIV-1_{LAI}. The genotype of the passaged virus was determined as described above.

Drug susceptibility assays. NRTI susceptibility was determined in P4/R5 cells as described previously (Parikh, U. M., et al. 2005. Antimicrob. Agents Chemother. 49:1139-44). Briefly, threefold dilutions of inhibitor were added to P4/R5 cells in triplicate and cells were infected with an amount of virus that produced 100 relative units of light (RLU) in no drug, virus control wells. After 48 hours, the cells were lysed (Gal-Screen; Tropix/Applied Biosystems, Foster City, Calif.) and the RLU was measured using a ThermoLabSystems luminometer (Waltham, Mass.). The IC₅₀ and fold-resistance were calculated as described above. IC₅₀ values from at least three independent experiments were log₁₀ transformed and compared for statistically significant differences using the two-sample Student's t test.

Clonal analysis of HIV-1 RT for mutation linkage. The entire sequence of HIV-1 RT from passaged viruses was RT-PCR amplified using RT forward and RT reverse primers (defined above), and the PCR product was cloned into the TOPO TA® cloning vector (Invitrogen, Carlsbad, Calif.). After transformation into Escherichia coli TOP10 competent cells, clones containing the correct insert were identified through blue-white screening. DNA from clones was purified and sequenced as described above.

Construction of mutant recombinant HIV-1. Mutant recombinant plasmid clones of virus were generated by oligonucleotide site-directed mutagenesis as described previously (Shi, C., and J. W. Mellors. 1997. A recombinant retroviral system for rapid in vivo analysis of human immunodeficiency virus type 1 susceptibility to reverse transcriptase inhibitors. Antimicrob. Agents Chemother. 41:2781-5), using the p6HRT-MO plasmid. p6HRT-MO contains the entire RT and protease coding sequence as previously described (Le Grice, S. F., and F. Gruninger-Leitch. 1990. Rapid purification of homodimer and heterodimer HIV-1 reverse transcriptase by metal chelate affinity chromatography. Eur. J. Biochem. 187:307-14) and four silent restriction sites (Xma I, Mlu I, Xba I, and NgoM IV from the 5′ to 3′ end of RT at codons 14, 358, 490 and 554, respectively). After site-directed mutagenesis (QIAamp kit, QIAGEN, Valencia, Calif.), the mutated RT was ligated into pxxHIV-1_{LAI} MO that contains the entire genome of HIV-1_{LAI} and the same silent restriction sites as p6HRT-MO. Infectious virus was generated by electroporating the mutated xxHIV-1_{LAI} MO plasmid into MT-2 cells as described above. All mutations in recombinant viruses were confirmed by full-length sequencing of the entire RT coding region.

Assays of replication capacity and replication kinetics. The p24 (ng/ml) of each viral stock was determined by ELISA (Alliance HIV-1 p24 ELISA kit, PerkinElmer, Wellesley, Mass.) and single-cycle replication capacity was measured by adding 10 ng of viral p24 to 5×10⁴ P4/R5 cells in a 96 well plate (6 wells per virus). After 48 hours, the cells were lysed and the RLU was measured as described above. Mean RLU from three independent experiments were compared for statistically significant differences using the two-sample Student's t test. Multiple-cycle replication kinetics was determined in MT-2 cells. Virus (10 ng of p24) was added to 5×10⁶ MT-2 cells. After 2 hours, R10 was added to the infected MT-2 cells to give a final concentration of 1×10⁶ cells/ml. An initial aliquot was taken after the 2 hour infection as background, and samples of 0.5 ml were collected every day for 7 days. The cultures were replenished with 0.5 ml of R10 after each aliquot was harvested. The p24 (ng/ml) concentration of each aliquot was measured, and values from three independent experiments were compared for statistically significant differences using the two-sample Student's t test.

Visualization of three dimensional structure of HIV-1 RT. The Molecular Operating Environment (MOE™) (Chemical Computing Group Inc., Montreal, Quebec, Canada) was used to visualize structural images of RT bound to an RNA/DNA template/primer (T/P) (pdb access number 1HYS (Sarafianos, S. G., K. Das, C. Tantillo, A. D. Clark, Jr., J. Ding, J. M. Whitcomb, P. L. Boyer, S. H. Hughes, and E. Arnold. 2001. Crystal structure of HIV-1 reverse transcriptase in complex with a polypurine tract RNA:DNA. EMBO J 20:1449-61.).

Results

Selection of AZT-resistant virus. Two independent AZT selection experiments were conducted. One starting with wild-type HIV-1_{LAI} and the second starting with AZT HIV-1_{LAI} (encoding M41L/L210W/T215Y). Both viruses were serially passaged in MT-2 cells in increasing concentrations of AZT. After every 5 passages, AZT susceptibility was measured in a single-cycle viral replication assay in P4/R5 cells (described in Materials and Methods). Viral RNA was extracted, converted to cDNA and the entire coding region of RT (residues 1-560) was PCR amplified and sequenced to monitor the appearance of mutations. In the selection experiment starting with HIV-1_{LAI}, AZT susceptibility was reduced 66-fold by passage 35 and two polymerase domain TAMs were identified: D67N and K70R (Table 1).

TABLE 1
Selection of AZT-resistant virus starting with wild-type HIV-1_{LAI}

| | AZT | | | |
|---|---|---|---|---|
| | Concentration | IC₅₀ | Fold- | |
| Passage | (μM) | (μM) | Resistance^{a} | Mutations |
| 1 | 0.5 | 0.2 | 1.5 | ND^{b} |
| 10 | 0.5 | 0.3 | 1.6 | ND |
| 20 | 0.5 | 0.7 | 5.1 | ND |
| 25 | 1.0 | 0.7 | 4.1 | None |
| 30 | 2.0 | 3.0 | 26 | D67D/N, K70K/R |
| 35 | 2.0 | 10 | 66 | D67N, K70R |
| 40 | 4.0 | 10 | 86 | D67N, K70R, A371A/V, |
| | | | | Q509Q/L |
| 55 | 16 | 39 | 255 | D67N, K70R, T215T/I, |
| | | | | A371A/V, Q509Q/L |
| 60 | 32 | 56 | 489 | D67N, K70R, T215I, |
| | | | | A371A/V, Q509L |
| 65 | 32 | 810 | >16200 | D67N, K70R, T215I/F, |
| | | | | A371A/V, Q509L |

^{a}Fold-resistance compared to wild-type HIV-1_{LAI} passaged in parallel without AZT.
^{b}Not Done (ND)

By passage 40, the virus was ˜90-fold AZT-resistant and had acquired two novel mutations in RT: A371V and Q509L in the connection and RNase H domains of RT, respectively. By passage 60, T215I appeared and was then replaced by T215F by passage 65, increasing AZT resistance to ˜16,000-fold.

In the selection experiment starting with AZT HIV-1_{LAI}, AZT susceptibility was reduced>1,000-fold by passage 35 (Table 2).

TABLE 2
AZT selection starting with
HIV_{LAI} encoding M41L, K219W, T215Y (AZT^{R})

| | AZT | | | |
|---|---|---|---|---|
| | Concentration | IC₅₀ | Fold- | |
| Passage | (μM) | (μM) | Resistance^{a} | Mutations |
| 1 | 25 | 21 | 6.0 | ND^{b} |
| 10 | 50 | 34 | 15 | ND |
| 25 | 100 | 126 | 39 | M41L, D67D/N, L210W, |
| | | | | L214F, T215Y |
| 30 | 150 | 181 | 62 | M41L, D67D/N, L210W, |
| | | | | L214F, T215Y |
| 35 | 150 | >810 | >1332 | M41L, D67N, L210W, |
| | | | | L214F, T215Y |

^{a}Fold-resistance compared to wild-type HIV-1_{LAI} passaged in parallel without AZT.
^{b}Not Done (ND)

This decrease in AZT susceptibility was associated with the acquisition of two additional mutations in the DNA polymerase domain: D67N and L214F. Mutations in the connection or RNase H domains of RT were not detected.

Linkage analysis of mutations. To evaluate whether D67N, K70R, T215F, A371V and Q509L were selected on the same viral genome in the first selection experiment, the RT coding region from passage 65 virus was amplified by RT-PCR, cloned into the TOPO TA® prokaryotic vector and transformed into E. coli TOP10 cells. Plasmid DNA was isolated from twelve bacterial colonies and the full-length RT coding region was sequenced (Table 3). All 12 clones contained D67N, K70R and Q509L. Six of the clones had all 5 mutations and three clones contained T215I with D67N, K70R, A371V and Q509L. The remaining 3 clones contained D67N, K70R, Q509L and either T215I or T215F, but not A371V. Additional mutations that were identified included R358K in only 4 clones and F416Y in 3 clones (Table 3).

TABLE 3
D67N, K70R, T215I/F, A371V and Q509L are linked on the same genome

| | Clone Number | | | | | | | | | | | | % of |
|---|---|---|---|---|---|---|---|---|---|---|---|---|---|
| HIV_{LAI} | 1 | 2 | 3 | 4 | 5 | 6 | 7 | 8 | 9 | 10 | 11 | 12 | Clones |
| G18 | — | — | — | D | — | — | — | — | — | — | — | — | 8 |
| L26 | — | — | — | — | — | — | — | — | — | — | — | S | 8 |
| T39 | — | — | — | P | — | — | — | — | — | — | — | — | 8 |
| D67 | N | N | N | N | N | N | N | N | N | N | N | N | 100 |
| K70 | R | R | R | R | R | R | R | R | R | R | R | R | 100 |
| L73 | — | — | — | — | — | — | — | S | — | — | — | — | 8 |
| I94 | — | — | — | T | — | — | — | — | — | — | — | — | 8 |
| G99 | — | — | — | — | — | — | — | E | — | — | — | — | 8 |
| K104 | — | — | — | — | N | — | E | — | — | — | — | — | 8(N), 8(E) |
| S117 | — | — | — | — | L | — | — | — | — | — | — | — | 8 |
| V118 | — | — | — | I | — | — | — | — | — | — | — | — | 8 |
| D123 | — | G | — | — | — | — | — | — | — | — | — | — | 8 |
| G190 | — | — | — | — | — | Q | — | — | — | — | — | — | 8 |
| S191 | — | — | T | — | — | — | — | — | — | — | — | — | 8 |
| I202 | — | — | — | — | — | T | — | — | — | — | T | — | 16 |
| T215 | F | I | I | I | F | F | F | F | I | F | F | I | 42(I), 58(F) |
| E122 | — | — | — | — | — | — | — | — | G | — | — | — | 8 |
| Q242 | — | — | — | — | — | — | — | — | — | — | R | — | 8 |
| I288 | — | — | — | — | — | — | — | S | — | — | — | — | 8 |
| W337 | — | — | — | — | R | — | — | — | — | — | — | — | 8 |
| R358 | — | — | — | — | — | K | K | — | — | K | — | K | 33 |
| A371 | V | V | V | — | V | V | — | V | V | V | V | — | 75 |
| K385 | — | — | — | — | E | — | — | — | — | — | — | — | 8 |
| I393 | — | — | L | — | — | — | — | — | — | — | — | — | 8 |
| F416 | Y | — | — | — | — | — | — | — | Y | Y | — | — | 25 |
| I434 | — | — | — | — | M | — | — | — | — | — | — | — | 8 |
| A445 | — | — | — | — | — | V | — | — | — | — | — | — | 8 |
| L491 | — | — | — | R | — | — | — | — | — | — | — | — | 8 |
| R461 | — | — | — | — | K | — | — | — | — | — | — | — | 8 |
| N494 | — | — | — | — | — | — | — | — | S | — | — | — | 8 |
| A502 | T | — | — | — | — | — | — | — | — | — | — | — | 8 |
| Q509 | L | L | L | L | L | L | L | L | L | L | L | L | 100 |
| L517 | — | — | — | — | — | — | — | — | — | — | — | S | 8 |
| N519 | — | — | — | — | — | — | — | — | S | — | — | — | 8 |
| A534 | — | — | — | — | — | T | — | — | — | — | — | — | 8 |
| D549 | — | — | N | — | — | — | — | — | — | — | — | — | 8 |
| A554 | — | — | T | — | — | — | — | — | — | — | — | — | 8 |
| G555 | Q | — | — | — | — | — | — | — | — | — | — | — | 8 |
| G555 | — | — | K | — | — | — | — | — | — | — | — | — | 8 |

Drug susceptibility of recombinant viruses. To confirm the role of the A371V and Q509L mutations in AZT resistance, recombinant mutant viruses were generated by site-directed mutagenesis. Five mutant viruses were constructed that represent the appearance of mutations in the AZT selection experiment at passages 35, 40, 60 and 65 (Table 1). An additional 10 mutant viruses were generated to delineate the roles of A371V and Q509L alone and together with different combinations of TAMs (Table 4).

TABLE 4
AZT susceptibility of site-directed mutants

| | | Fold - | |
|---|---|---|---|
| Mutation | IC₅₀ (μM)^{a} | Resistance^{b} | p-value |
| Wild-type | 0.2 ± 0.1 | — | — |
| A371V | 0.2 ± 0.04 | 0.7 | 0.2 |
| Q509L | 0.3 ± 0.2 | 1.3 | 0.6 |
| A371V/Q509L | 0.3 ± 0.06 | 1.7 | 0.4 |
| 67N/70R^{c} | 1.1 ± 0.6 | 4.6 | <0.001 |
| 67N/70R/371V | 1.4 ± 0.5 | 6.4 | <0.001 |
| 67N/70R/509L | 3.0 ± 1.0 | 14 | <0.001 |
| 67N/70R/371V/509L | 9.1 ± 5.2 | 39 | <0.001 |
| 67N/70R/215I | 0.3 ± 0.2 | 1.3 | 0.5 |
| 67N/70R/215I/371V | 0.6 ± 0.3 | 2.6 | 0.07 |
| 67N/70R/215I/509L | 3.0 ± 2.2 | 14 | 0.004 |
| 67N/70R/215I/371V/509L | 9.4 ± 6.7 | 41 | <0.001 |
| 67N/70R/215F | 3.8 ± 2.1 | 18 | 0.002 |
| 67N/70R/215F/371V | 4.9 ± 2.9 | 22 | <0.001 |
| 67N/70R/215F/509L | 28 ± 17 | 128 | <0.001 |
| 67N/70R/215F/371V/509L | 203 ± 40 | 934 | <0.001 |

^{a}Mean ± standard deviation from at least three experiments.
^{b}Fold-resistance of mutants compared to wild-type.
^{c}Mutation combinations in bold were selected in vitro by AZT.

The A371V and Q509L mutations, alone or together, did not confer AZT resistance in the absence of TAMs. When the A371V mutation alone was added to viruses that contained different combinations of TAMs, AZT susceptibility was only marginally decreased (1.2- to 2-fold). By contrast, viruses that contained Q509L and different combinations of TAMs exhibited significantly greater resistance to AZT (3.0- to 11-fold). When both A371V and Q509L were combined with TAMs, the extent of AZT resistance was significantly greater (9- to 52-fold) compared with viruses that contained only one of the mutations or neither of them. Of note, there was only a small difference in AZT resistance between the D67N/K70R/A371V/Q509L mutant (39-fold) and the D67N/K70R/T215I/A371V/Q509L mutant (41-fold) (Table 4). Thus, the selective advantage of T215I was not obvious from these drug-susceptibility analyses.

Cross-resistance to other NRTIs. The effect of A371V and Q509L in combination with TAMs on cross-resistance to other NRTIs was also analyzed (Table 5). Statistically significant increases in cross-resistance to 3TC (p=0.047 and 0.014 for D67N/K70R/A371V/Q509L and D67N/K70R/T215F/A371V/Q509L, respectively) and abacavir (p=0.020 and 0.23, respectively) were noted in viruses that contained A371V and Q509L in combination with TAMs compared with those that contained only TAMs. Viruses that contained TAMs and A371V and Q509L also exhibited a trend toward decreased susceptibility to tenofovir (p=0.10 and 0.058, respectively), but not to d4T or ddI (Table 5).

TABLE 5
Cross-resistance of site-directed mutants to NRTIs

| | IC₅₀ (Fold-Resistance)^{a} | | | | |
|---|---|---|---|---|---|
| | lamivudine | abacavir | tenofovir | stavudine | didanosine |
| Mutation in HIV_{LAI} RT | (3TC) | (ABC) | (TNV) | (d4T) | (ddI) |
| Wild-type | 0.5 ± 0.1 | 6.4 ± 0.5 | 3.3 ± 0.9 | 7.2 ± 0.6 | 4.2 ± 0.9 |
| D67N/K70R | 1.0 ± 0.3 (1.9)^{c} | 7.9 ± 0.4 (1.2)^{c} | 4.9 ± 1.5 (1.5) | 10 ± 3.1 (1.4) | 5.0 ± 0.9 (1.2) |
| D67N/K70R/A371V/Q509L | 2.7 ± 1.4 (5.2)^{c,d} | 13 ± 2.5 (2.0)^{c,d} | 7.3 ± 1.3 (2.2)^{c} | 9.6 ± 2.9 (1.3) | 4.9 ± 0.9 (1.2) |
| D67N/K70R/T215F | 3.8 ± 0.59 (7.0)^{b} | 15 ± 1.5 (2.4)^{b} | 4.9 ± 0.7 (1.5) | 17 ± 4.6 (2.4)^{c} | 6.8 ± 0.3 (1.6)^{c} |
| D67N/K70R/T215F/A371V/Q509L | 7.5 ± 1.8 (15)^{b,e} | 19 ± 4.5 (3.0)^{c} | 9.0 ± 3.5 (2.7)^{c} | 14 ± 4.4 (2.0)^{c} | 6.6 ± 0.4 (1.6)^{c} |

^{a}Mean ± SD is from at least 3 independent experiments. Fold-resistance compared to wild-type in parentheses.
^{b}IC₅₀ is significantly different from wild-type, p < 0.001.
^{c}IC₅₀ is significantly different from wild-type, p < 0.05.
^{d}IC₅₀ is significantly different from D67N/K70R, p < 0.05.
^{e}IC₅₀ is significantly different from D67N/K70R/T215F, p < 0.05.

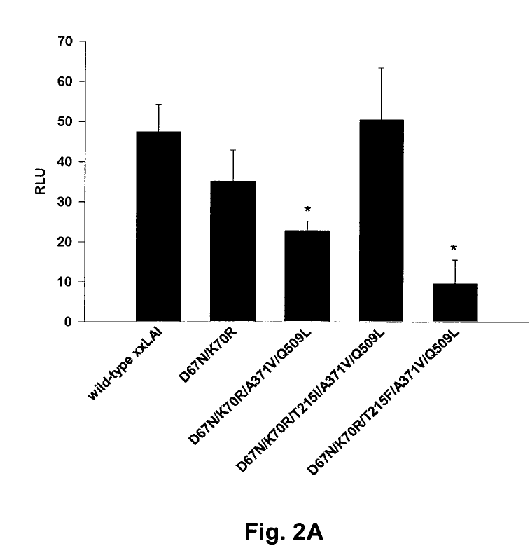

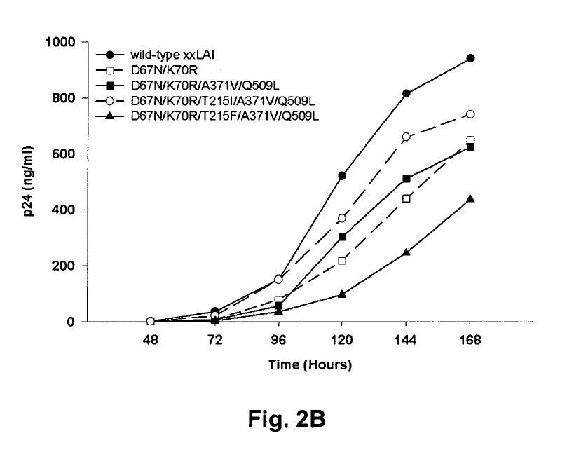

Replication capacity and replication kinetics of mutant viruses. Since the selective advantage of T215I was not evident from the drug-susceptibility analyses (Table 4), we next assessed replication capacity and kinetics of the 4 recombinant viruses with RT sequences identical to those in viruses from passages 35, 40, 60 and 65. Replication capacity was assessed in a single-cycle assay in P4/R5 cells and replication kinetics was assessed using a multiple-cycle assay in MT-2 cells. Cells were infected with a standard inoculum (10 ng of p24) of each virus. FIG. 2A shows that the replication capacity of the D67N/K70R/A371V/Q509L mutant was reduced to 48% of wild-type virus. This loss in replication capacity, however, was restored to wild-type levels by the addition of the T215I mutation. By contrast, the T215F mutation reduced the replication capacity of the D67N/K70R/T215F/A371V/Q509L virus to 20% of wild-type virus. This reduction in replication capacity from the T215F mutation was associated with significantly greater AZT resistance (Table 4), illustrating a trade-off between replication capacity and resistance. Similar results were observed in replication kinetic assays carried out in MT-2 cells over a 7 day period (FIG. 2B). Specifically, the replication of the D67N/K70R and D67N/K70R/A371V/Q509L mutant viruses was reduced 46% and 37% on day 6, respectively, compared with wild-type virus. The impaired replication of the D67N/K70R/A371V/Q509L virus was restored to levels similar to wild-type by the T215I mutation. As noted above, the T215F mutation markedly impaired viral replication.

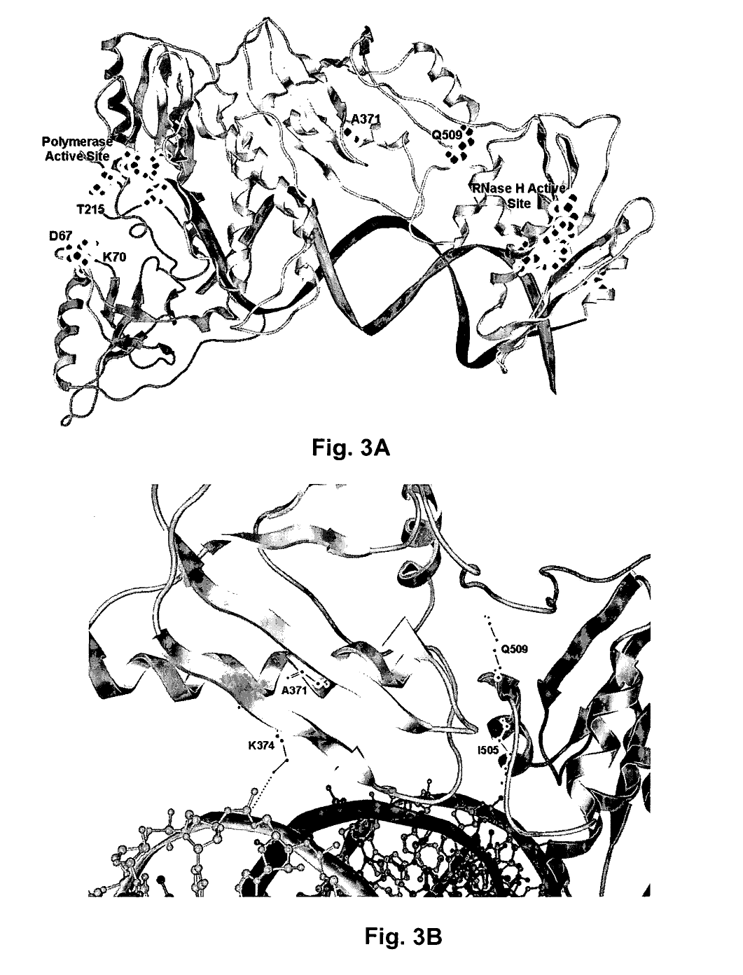

Location of residues A371 and Q509 in RT Analysis of a crystal structure of HIV-1 RT in complex with an RNA/DNA polypurine tract T/P substrate reveals that both A371 and Q509 are located near the T/P DNA binding tract (FIGS. 3A and 3B). A371 is 2.8 Å from K374, the side-chain of which interacts with the phosphate backbone of the RNA template strand (grey ribbon) through a hydrogen bond (FIG. 3B). Q509 is close to the RNase H primer grip, in particular, residue 1505. The RNase H primer grip of HIV-1 RT contacts the DNA primer strand (black ribbon) and positions the template strand near the RNase H active site, influencing RNase H cleavage efficiency and specificity (Sarafianos, S. G., et al. 2001. EMBO J 20:1449-61).

Discussion

In this study it is shown that AZT selects novel mutations in RT, namely A371V in the connection domain and Q509L in the RNase H domain, that increase AZT resistance by ˜50-fold with the “classic” TAMs in the polymerase domain: D67N, K70R and T215I/F. This provides the first definitive virologic evidence that mutations in both the connection and RNase H domains are selected by AZT for HIV-1 resistance. In addition, it is shown that these mutations when combined with TAMs confer greater cross-resistance to 3TC and ABC, with a trend toward greater TNV resistance.

The only mutations that arose during the selection that started with AZT-resistant virus encoding the TAMs M41L, L210W and T215Y were D67N and L214F in the polymerase domain (Table 2), which increased AZT resistance by >1,332-fold at passage 35. No mutations were detected in the connection or RNase H domains. This indicates that very high-level AZT resistance is possible with mutations restricted to the polymerase domain, and suggests that A371V and Q509L mutations are only advantageous in certain TAM backgrounds. The construction of additional recombinant viruses is in progress to verify this.

Other evidence suggests that mutations outside of the polymerase domain of HIV-1 RT are involved in resistance to NRTI. For example, Nikolenko et al recently demonstrated that mutations that reduce RNase H activity, such as D549N and H539N, increase AZT resistance (Nikolenko, G. N., et al. 2005. Proc. Natl. Acad. Sci. USA 102:2093-8), but these mutations have not been identified in viruses from antiretroviral-experienced patients nor have they been selected by AZT in vitro. Initial analyses of clinical samples, however, have identified mutations in the connection and RNase H domains of RT that can increase AZT resistance (Galli, R., B. Wynhoven, B. Sattha, G. Tachedjian, and P. Harrigan. 2004. Beyond codon 240: Mutations in the HIV-1 reverse transcriptase selected after exposure to antiretrovirals. eJIAS 1; Gupta, S., S. Fransen, E. E. Paxinos, W. Huang, E. Stawiski, C. J. Petropoulos, and N. T. Parkin. 2006. Infrequent occurrence of mutations in the C-terminal region of reverse transcriptase modulates susceptibility to RT inhibitors. Antivir. Ther. 11:S143; and Nikolenko, G. N., K. A. Franenberry, S. Palmer, F. Maldarelli, J. W. Mellors, J. M. Coffin, and V. K. Pathak. 2006. The HIV-1 reverse transcriptase connection domain from treatment-experienced patients contributes to AZT resistance. Antivir. Ther. 11:S142). For example, mutations G335C, N3481 and A360I reduce AZT susceptibility 30-, 35- and 30-fold, respectively; when present with “classic” TAMs (Gupta, S., S. et al. 2006. Antivir. Ther. 11:S143 and Nikolenko, G. N., et al. 2006. Antivir. Ther. 11:S142). In addition, a polymorphism at RT amino acid 333 (G to E) has been observed in samples from patients on combination therapy with AZT and 3TC (Kemp, S. D., C. Shi, S. Bloor, P. R. Harrigan, J. W. Mellors, and B. A. Larder. 1998. A novel polymorphism at codon 333 of human immunodeficiency virus type 1 reverse transcriptase can facilitate dual resistance to zidovudine and L-2′,3′-dideoxy-3′-thiacytidine. J. Virol. 72:5093-8). The G333E polymorphism counteracts the increase in AZT sensitivity of virus with the 3TC resistance mutation, M184V (Tisdale, M., S. D. Kemp, N. R. Parry, and B. A. Larder. 1993. Rapid in vitro selection of human immunodeficiency virus type 1 resistant to 3′-thiacytidine inhibitors due to a mutation in the YMDD region of reverse transcriptase. Proc. Natl. Acad. Sci. USA 90:5653-6).

Several retrospective statistical analyses of clinical genotype databases have also identified mutations in the connection and RNase H domains of RT that appear more frequently in samples from antiretroviral-experienced patients than antiretroviral-naïve patients (Chen, L., A. Perlina, and C. J. Lee. 2004. Positive selection detection in 40,000 human immunodeficiency virus (HIV) type 1 sequences automatically identifies drug resistance and positive fitness mutations in HIV protease and reverse transcriptase. J. Virol. 78:3722-32; Galli, R., B. et al. 2004. eJIAS 1; and Rhee, S. Y., M. J. Gonzales, R. Kantor, B. J. Betts, J. Ravela, and R. W. Shafer. 2003. Human immunodeficiency virus reverse transcriptase and protease sequence database. Nucleic Acids Res. 31:298-303). However, the roles of these mutations in NRTI resistance have not been proven. The A371V and Q509L mutations have been identified in patient genotypes in the Stanford HIV Drug Resistance Database (Rhee, S. Y., et al. 2003. Nucleic Acids Res. 31:298-303), and our preliminary analysis of this database reveals that patients treated with AZT show an increase in frequency of several mutations in the C-terminus of RT (amino acids 350-560). For example, A371V was detected in 5.6% of 160 samples from treatment-naïve individuals, and in 10.9% of 91 samples from patients treated with AZT monotherapy. Another mutation at codon 371 (A to T) is also seen at 2.1% frequency in AZT monotherapy samples. In addition, A371V is associated with mutations at T215 (Y/F/I/S) in 77% of the AZT monotherapy samples, and with 46%, 23%, 31%, 23% and 15% of the samples with M41L, D67N, K70R, L210W and K219Q, respectively. Only 16 full-length sequences (to codon 560) are available in the Stanford database, and none of these have mutations at codon 509. Additional full-length RT sequences from patients who have received AZT therapy are being generated to examine the RNAse H domain including codon 509.

Two phenotypic mechanisms of NRTI resistance have been proposed. The first is NRTI discrimination and involves mutations in RT (such as K65R, K70E, L74V, Q151M and M184V) that enable RT to preferentially incorporate the natural dNTP substrate versus the NRTI-TP (Boyer, P. L., S. G. Sarafianos, E. Arnold, and S. H. Hughes. 2002. The M184V mutation reduces the selective excision of zidovudine 5′-monophosphate (AZTMP) by the reverse transcriptase of human immunodeficiency virus type 1. J. Virol. 76:3248-56; Boyer, P. L., C. Tantillo, A. Jacobo-Molina, R. G. Nanni, J. Ding, E. Arnold, and S. H. Hughes. 1994. Sensitivity of wild-type human immunodeficiency virus type 1 reverse transcriptase to dideoxynucleotides depends on template length; the sensitivity of drug-resistant mutants does not. Proc. Natl. Acad. Sci. USA 91:4882-6 Sarafianos, S. G., K. Das, A. D. Clark, Jr., J. Ding, P. L. Boyer, S. H. Hughes, and E. Arnold; 1999. Lamivudine (3TC) resistance in HIV-1 reverse transcriptase involves steric hindrance with beta-branched amino acids. Proc. Natl. Acad. Sci. USA 96:10027-32; and Sluis-Cremer, N., P. Argoti Torres, J. Grzybowski, U. Parikh, and J. Mellors. 2006. Presented at the 13th Conference on Retroviruses and Opportunistic Infections, Denver, Colo.). The second mechanism has been termed NRTI excision associated with TAMs. The available biochemical evidence suggests that TAMs increase the ability of HIV-1 RT to phosphorolytically excise AZT-monophosphate (AZT-MP) from the chain-terminated T/P (Boyer, P. L., S. G. Sarafianos, E. Arnold, and S. H. Hughes. 2001. Selective excision of AZTMP by drug-resistant human immunodeficiency virus reverse transcriptase. J. Virol. 75:4832-42 and Sarafianos, S. G., A. D. Clark, Jr., K. Das, S. Tuske, J. J. Birktoft, P. Ilankumaran, A. R. Ramesha, J. M. Sayer, D. M. Jerina, P. L. Boyer, S. H. Hughes, and E. Arnold. 2002. Structures of HIV-1 reverse transcriptase with pre- and post-translocation AZTMP-terminated DNA. EMBO J 21:6614-24). Because A371V and Q509L were selected in combination with TAMs and do not confer resistance to AZT alone, we hypothesize that these mutations enhance the RT-mediated excision reaction. Furthermore, the relatively small increases in cross-resistance to 3TC, ABC and TNV in viruses having the A371V and Q509L mutations suggest that these mutations may be largely specific for AZT.

Analysis of the crystal structure of RT bound to an RNA/DNA T/P showed that A371V and Q509L reside close to the DNA-binding tract in RT (FIG. 3). This suggests that the mutations may affect either T/P interactions (in the case of A371V and Q509L) or RNase H activity (in the case of Q509L). With regard to the latter, several studies have clearly demonstrated that mutations in the RNase primer grip can significantly impact the rates and efficiency of RNase H cleavage (Julias, J. G., M. J. McWilliams, S. G. Sarafianos, W. G. Alvord, E. Arnold, and S. H. Hughes. 2003. Mutation of amino acids in the connection domain of human immunodeficiency virus type 1 reverse transcriptase that contact the template-primer affects RNase H activity. J. Virol. 77:8548-54 and Rausch, J. W., D. Lener, J. T. Miller, J. G. Julias, S. H. Hughes, and S. F. Le Grice. 2002. Altering the RNase H primer grip of human immunodeficiency virus reverse transcriptase modifies cleavage specificity. Biochemistry 41:4856-65). Mechanistic studies are currently underway to define the biochemical mechanisms by which A371V and Q509L increase AZT resistance.

Because there was only a small difference between the IC₅₀ values of viruses with D67N/K70R/A371V/Q509L and D67N/K70R/T215I/A371V/Q509L (Table 4), replication capacity and kinetics were performed to determine whether the T215I mutation affected viral replication capacity/kinetics. Single-cycle and multiple-cycle replication assays clearly showed that the T215I mutation restored replication capacity and kinetics of the D67N/K70R/T215I/A371V/Q509L mutant to wild-type levels (FIGS. 2A and 2B). This likely explains why the T215I mutant emerged without having a significant impact on AZT resistance. The T215I mutation was subsequently replaced by T215F at higher AZT selective concentrations. This replacement is likely explained by the T215F mutation conferring ˜25-fold greater AZT resistance than T215I, but at the cost of reduced replication capacity and kinetics in the absence of AZT.

We claim:

1. A method of determining the presence of a nucleoside reverse transcriptase inhibitor-resistant Human Immunodeficiency Virus-1 (HIV-1) virus particle in a biological sample, comprising identifying the presence in the sample of a point mutation at codon Q509 of an HIV-1 reverse transcriptase.

2. The method of claim 1, wherein the point mutation is Q509L.

3. The method of claim 1, wherein the nucleoside reverse transcriptase inhibitor is one of 3′-azido-3′-deoxythymidine, 2,3-didehydro-2,3-dideoxythymidine, didanosine, zalcitabine, lamivudine, abacavir and emtricitabine.

4. The method of claim 3, wherein the nucleoside reverse transcriptase inhibitor is 3′-azido-3′-deoxythymidine.

5. The method of claim 1, further comprising determining the presence in the biological sample of a point mutation at one or more of codons 67, 70, 215 and 371 of an HIV-1 reverse transcriptase.

6. The method of claim 5, further comprising determining the presence in the biological sample of one or more of the point mutations M41L, D67N, K70R, T215I, T215F, T215Y and A371V in an HIV-1 reverse transcriptase.

7. The method of claim 5, comprising determining the presence in the biological sample of the point mutations D67N, K70R and Q509L in an HIV-1 reverse transcriptase.

8. The method of claim 5, further comprising determining the presence in the biological sample of the point mutation A371V in an HIV-1 reverse transcriptase.

9. The method of claim 1, further comprising determining the presence in the biological sample of a thymidine analog mutation (TAM) in an HIV-1 reverse transcriptase.

10. The method of claim 1, further comprising determining the presence in the biological sample of the point mutation T215I in an HIV-1 reverse transcriptase.

11. The method of claim 1, further comprising determining the presence in the biological sample of the point mutation T215F in an HIV-1 reverse transcriptase.

12. The method of claim 1, further comprising determining the presence in the biological sample of the point mutations D67N and K70R in an HIV-1 reverse transcriptase.

13. The method of claim 12, further comprising determining the presence in the biological sample of the point mutation T215I in an HIV-1 reverse transcriptase.

14. The method of claim 12, further comprising determining the presence in the biological sample of the point mutation T215F in an HIV-1 reverse transcriptase.

15. The method of claim 12, further comprising determining the presence in the biological sample of the point mutation A371V in an HIV-1 reverse transcriptase.

16. The method of claim 15, further comprising determining the presence in the biological sample of the point mutation T215I in an HIV-1 reverse transcriptase.

17. The method of claim 15, further comprising determining the presence in the biological sample of the point mutation T215F in an HIV-1 reverse transcriptase.

18. The method of claim 1, further comprising determining the presence in the biological sample of the point mutation M41L in an HIV-1 reverse transcriptase.

19. The method of claim 1, comprising preparing cDNA from HIV-1 RNA in the sample and sequencing at least a portion of the cDNA or an amplification product thereof to determine the presence of the point mutation in the reverse transcriptase.

20. The method of claim 19, wherein a portion of the cDNA comprising a sequence encoding codon 509 of the reverse transcriptase protein is amplified and sequenced.

21. The method of claim 20, wherein a portion of the cDNA comprising a sequence encoding codons 41 through 509 of the reverse transcriptase protein is amplified and sequenced.

22. The method of claim 1, wherein the point mutation is identified by one or more of: sequencing of a cDNA or an amplification product thereof, allele-specific PCR, Oligonucleotide Ligation assay, clonal analysis and Single Genome Sequencing.

23. An isolated nucleic acid comprising at its 3′ terminus one of the sequences: 5′-ttcaagcaca-3′ (SEQ ID NO: 3, nucleotides 27-36), 5′-ttcaagcact-3′ (SEQ ID NO: 4, nucleotides 27-36), 5′-ttatctggta-3′ (SEQ ID NO: 5, nucleotides 31-40) or 5′-ttatctggtt-3′ (SEQ ID NO: 6, nucleotides 31-40).

24. The isolated nucleic acid of claim 23, comprising at its 3′ terminus 10 or more contiguous nucleotides of the 3′ end of one of the sequences:
(SEQ ID NO: 3)
5′-agactcacaatatgcattaggaatcattcaagcaca-3′,
(SEQ ID NO: 4)
5′-agactcacaatatgcattaggaatcattcaagcact-3′,
(SEQ ID NO: 5)
5′-attatttgattgactaactctgattcacttttatctggta-3′,
or
(SEQ ID NO: 6)
5′-attatttgattgactaactctgattcacttttatctggtt-3′.

24. The nucleic acid of claim 22, wherein the nucleic acid is fluorescently-labeled.
